(19) United States
(12) Patent Application Publication
(10) Pub. No.: US 2018/0168857 A1
(43) Pub. Date: Jun. 21, 2018

(54) Intra-Oral Orthodontic Cooling Pack

(71) Applicant: Jennifer L. Connelly, Katonah, NY (US)

(72) Inventor: Jennifer L. Connelly, Katonah, NY (US)

(21) Appl. No.: 15/842,406

(22) Filed: Dec. 14, 2017

Related U.S. Application Data
(60) Provisional application No. 62/434,681, filed on Dec. 15, 2016.

(51) Int. Cl. A61F 7/12; A61F 7/02; A61C 19/06

(52) U.S. Cl. CPC A61F 7/12; A61F 2007/0017; A61C 19/06; A61F 7/02

(57) ABSTRACT

An intra-oral orthodontic cooling pack device for providing a pain relief, numbing, and cooling effect to the gums and teeth. The intra-oral orthodontic cooling pack includes a plurality of chambers interconnected pivotally wherein the chambers each define an interior volume and can conform to a curved surface, such as an upper or lower gum line. A first attachment mechanism extends from a first end of the plurality of chambers and a second attachment mechanism extending from a second end of the plurality of chambers, wherein the first and second attachment mechanism removably secure the intra-oral orthodontic cooling pack to a support, such as a bracket found on orthodontic braces.

### CROSS REFERENCE TO RELATED APPLICATIONS

This application claims the benefit of U.S. Provisional Application No. 62/434,681 filed on Dec. 15, 2016. The above identified patent application is herein incorporated by reference in its entirety to provide continuity of disclosure.

### BACKGROUND OF THE INVENTION

The present invention relates to a cooling pack for people with orthodontic braces. More specifically, the present invention provides an intra-oral orthodontic cooling pack adapted to be secured to a support to rest against a wearer's gums.

Many children and adults wear orthodontic braces in order to align and straighten teeth. Braces typically use a tension wire attached to brackets placed on each tooth in order to slowly reposition the teeth within the mouth. The constant tension on the wearer's teeth often leads to pain and discomfort in the teeth and gums. Each time the braces are tightened to add more tension, pain and discomfort can increase significantly.

Devices have been disclosed in the known art that relate to insertable oral pain relief devices. However, these devices have several known drawbacks. These devices fail to provide attachment mechanisms removably securable to a support, such as a bracket found on orthodontic braces. Furthermore, these devices fail to provide a plurality of chambers adapted to conform to a curved surface.

In light of the devices disclosed in the known art, it is submitted that the present invention substantially diverges in design elements from the prior art and consequently it is clear that there is a need in the art for an improvement to existing oral pain relief devices. In this regard the instant invention substantially fulfills these needs.

### SUMMARY OF THE INVENTION

In view of the foregoing disadvantages inherent in the known types of oral pain relief devices now present in the prior art, the present invention provides an intra-oral orthodontic cooling pack wherein the same can be utilized for providing convenience for the user when relieving oral pain. The intra-oral orthodontic cooling pack comprises a plurality of chambers, pivotally connected to each other, having a first and second end, wherein the chambers each define an interior volume. A first attachment mechanism extends from the first end and a second attachment mechanism extends from the second end, wherein the attachment mechanisms are adapted to removably secure to a support surface, such as a bracket found on orthodontic braces.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the intra-oral orthodontic ice pack.

FIG. 2 shows a cross section view of an embodiment of the intra-oral orthodontic ice pack.

FIG. 3 shows a side view of an embodiment of the intra-oral orthodontic ice pack in use.

FIG. 4 shows a front view of an embodiment of the intra-oral orthodontic ice pack in use.

FIG. 5 shows a perspective view of an alternative embodiment of the intra-oral orthodontic ice pack.

### DETAILED DESCRIPTION OF THE INVENTION

Reference is made herein to the attached drawings. Like reference numerals are used throughout the drawings to depict like or similar elements of the intra-oral orthodontic cooling pack. The figures are intended for representative purposes only and should not be considered to be limiting in any respect.

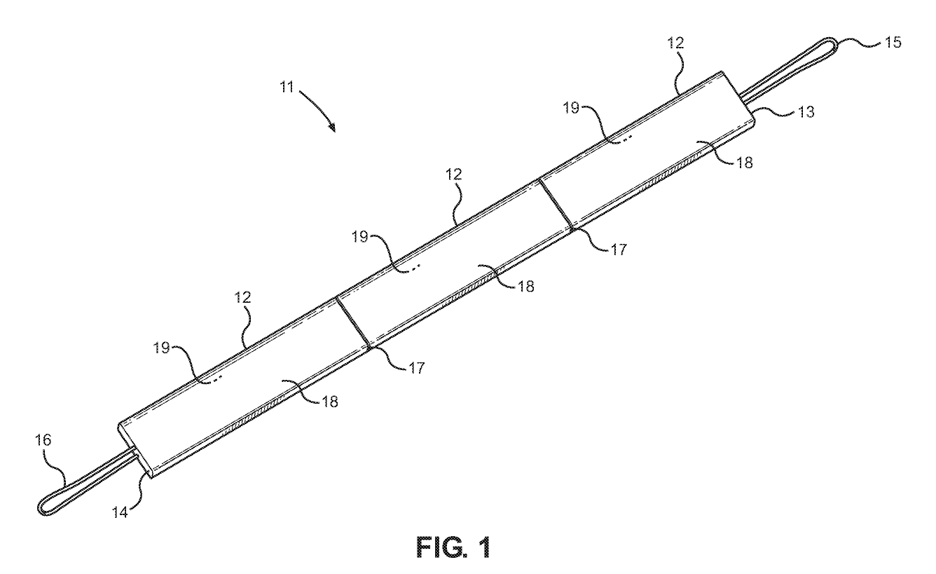

Referring now to FIG. 1, there is shown a perspective view of an embodiment of the intra-oral orthodontic ice pack. The intra-oral orthodontic cooling pack 11 comprises a plurality of chambers 12 pivotally connected to each other about a gap 17. In the illustrated embodiment, the gap 17 is formed between both an upper surface 18 and a lower surface 19 of the adjacent chambers, such that the intra-oral orthodontic cooling pack 11 can be flexed equally in either direction. In the illustrated embodiment, the gap 17 is continuously present when the intra-oral orthodontic cooling pack 11 is in both the flexed and unflexed positions. In the illustrated embodiment, the gap 17 comprises a pivot axis, however, in alternative embodiments, the gap 17 comprises greater lengths to provide increased flexibility to conform to a curved surface. The plurality of chambers 12 are aligned side-by-side, such that the intra-oral orthodontic cooling pack 11 is elongated.

In some embodiments, the plurality of chambers 12 comprise an elastic material adapted to provide a tension force to assist in holding the intra-oral orthodontic cooling pack 11 in place. The plurality of chambers 12, in some embodiments, comprise a flexible material such that each chamber is configured to bend to allow increased surface area contact to a curved surface. In the illustrated embodiments, the plurality of chambers 12 comprise a substantially rectangular shape, however, in alternative embodiments, the shape of the plurality of chambers 12 is configured to conform to the shape of a curved surface.

In the illustrated embodiment, the plurality of chambers 12 have a first end 13 and a second end 14. Extending from the first end 13 is a first attachment mechanism 15 and extending from the second end 14 is a second attachment mechanism 16. In the illustrated embodiment, the first and second attachment mechanisms 15, 16 comprise a loop. However, in alternative embodiments, the first and second attachment mechanisms 15, 16 comprise any suitable mechanism that is adapted to removably secure to a support structure, such as a bracket found in orthodontic braces. In some embodiments, the first and second attachment mechanisms 15, 16 comprise an elastic material adapted to provide a tension force to assist in holding the intra-oral orthodontic cooling pack 11 in place.

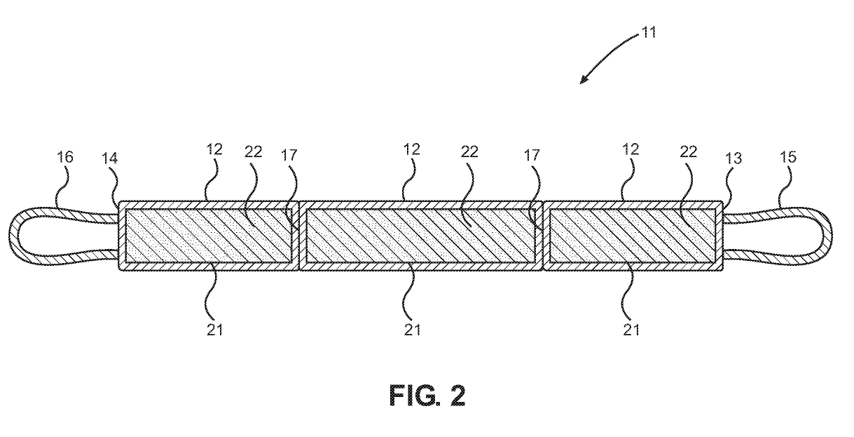

Referring now to FIG. 2, there is shown a cross-sectional view of an embodiment of the intra-oral orthodontic ice pack. The plurality of chambers 12 each define an internal volume 21. Contained within the internal volume 21 is a non-toxic coolant 22. In the illustrated embodiment, the non-toxic coolant is adapted to freeze into a solid or quasi-solid state. In this way, the non-toxic coolant 22 can flex with the plurality of chambers 12 when in the quasi-solid state to conform to the shape of a user's mouth, thereby increasing the surface area through which to transfer heat. In some embodiments, the non-toxic coolant 22 comprises a gel.

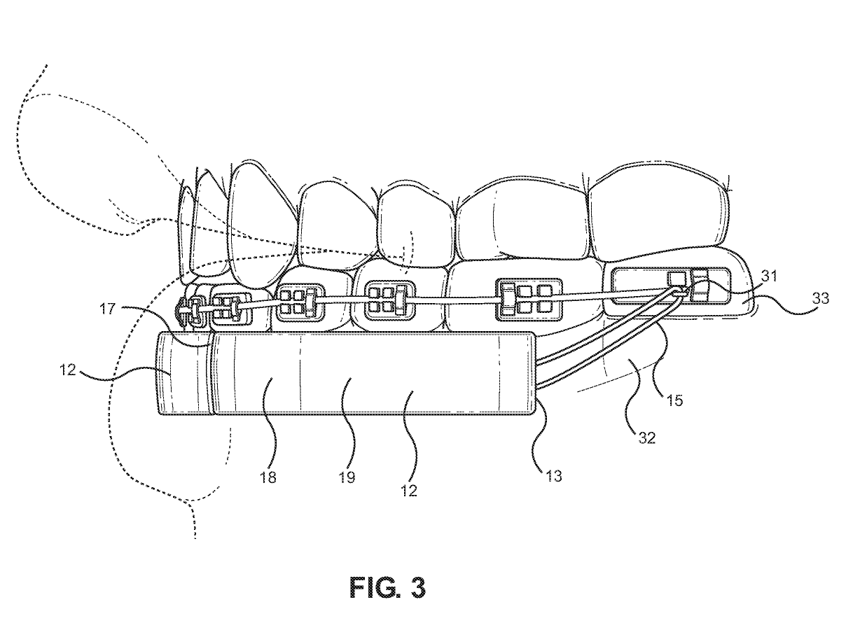

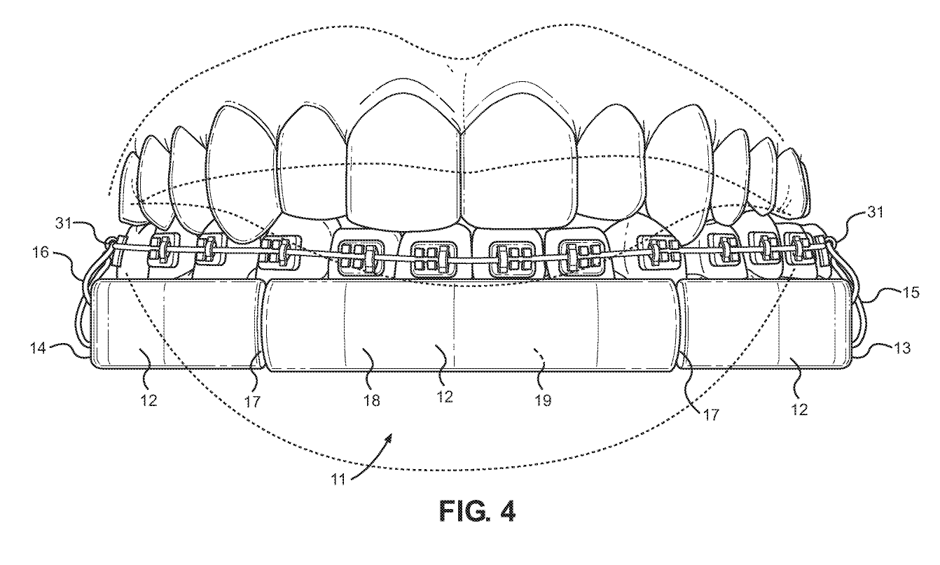

Referring now to FIGS. 3 and 4, there is shown a side view of an embodiment of the intra-oral orthodontic ice pack in use and a front view of an embodiment of the intra-oral orthodontic ice pack in use, respectively. In operation, the plurality of chambers 12 are positioned parallel to a gum line 32. In some embodiments, the plurality of chambers 12 are not configured to extend beyond a rear molar 33. In the illustrated embodiment, the first attachment mechanism 15 is removably secured to the orthodontic bracket 31. This, in tandem with the opposing second attachment mechanism 16 being removably secured to another orthodontic bracket 31, creates a tension force that acts to hold the intra-oral orthodontic cooling pack 11 in place. In some embodiments, the intra-oral orthodontic cooling pack includes the orthodontic bracket 31.

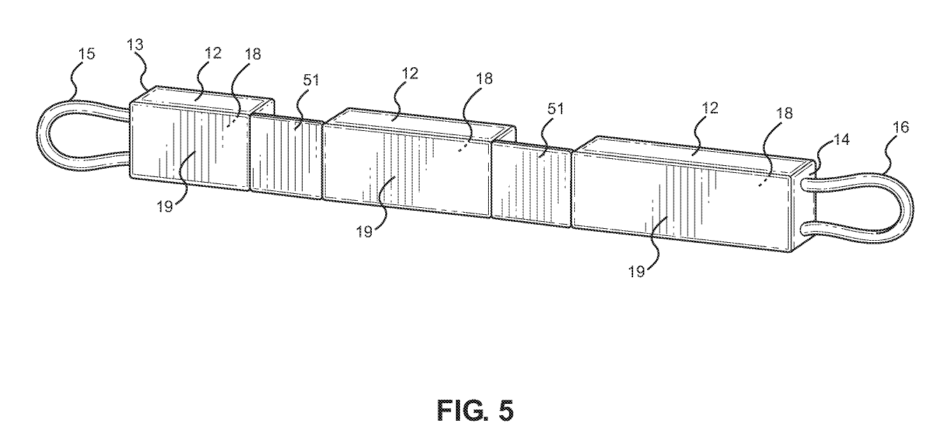

Referring now to FIG. 5, there is shown a perspective view of an alternate embodiment of the intra-oral orthodontic ice pack. In the illustrated embodiment, the plurality of chambers 12 are disposed along the length of a planar member 51. The length of planar member 51 comprises the same length or greater length than the sum of the lengths of the plurality of chambers 12. In this way, at greater lengths relative to the sum of the plurality of chambers 12, this provides increased flexibility to the intra-oral orthodontic ice pack 11. The planar member 51 is flexible and elastic allowing it to conform to the shape of a curved surface and provide a tension force to assist in holding the intra-oral orthodontic ice pack 11 in place. In the illustrated embodiment, the planar member 51 is configured to provide an even distribution of coolness across the gum line 32.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) An intra-oral orthodontic cooling pack, comprising:
a plurality of chambers, pivotally connected to each other, having a first and a second end, wherein the plurality of chambers each define an interior volume and are configured to rest between a user's lip and gum line.
a first attachment mechanism extending from the first end and a second attachment mechanism extending from the second end, wherein the first and second attachment mechanisms are adapted to removably secure to a support surface.

2) The intra-oral orthodontic cooling pack of claim 1, wherein the plurality of chambers, the first attachment mechanism, and the second attachment mechanism comprise the same material.

3) The intra-oral orthodontic cooling pack of claim 1, wherein the plurality of chambers comprise an elastic material configured to allow the chambers to stretch to provide a tension force.

4) The intra-oral orthodontic cooling pack of claim 1, wherein the plurality of chambers comprise a flexible material configured to allow the chambers to conform to a curved surface.

5) The intra-oral orthodontic cooling pack of claim 1, wherein the plurality of chambers contain a non-toxic coolant capable of being frozen into a solid or quasi-solid state.

6) The intra-oral orthodontic cooling pack of claim 3, wherein the non-toxic coolant is a gel.

7) The intra-oral orthodontic cooling pack of claim 1, wherein the plurality of chambers are disposed such that spaces are defined therebetween to allow the plurality of chambers to conform to a curved surface.

8) The intra-oral orthodontic cooling pack of claim 1, wherein the first and second attachment mechanisms comprise a loop.

9) An intra-oral orthodontic cooling pack, comprising:
a planar member having a first and a second end, wherein the planar member is elastic and flexible such that the planar member is configured to provide a tension force and allow the planar member to conform to a curved surface;
a plurality of chambers defining an interior volume disposed along the length of the planar member;
wherein the planar member and the plurality of chambers are configured to rest between a user's lip and gum line.
a first attachment mechanism extending from the first end and a second attachment mechanism extending from the second end, wherein the attachment mechanisms are adapted to removably secure to a support surface.

10) The intra-oral orthodontic cooling pack of claim 9, wherein the chambers contain a non-toxic coolant capable of being frozen into a solid or quasi-solid state.

11) The intra-oral orthodontic cooling pack of claim 9, wherein the chambers comprise the same flexible material as the planar member, to allow the chambers to conform to a curved surface.

12) The intra-oral orthodontic cooling pack of claim 9, wherein the chambers are disposed along the planar member such that spaces are defined therebetween to allow the planar member to conform to a curved surface.
